(19) United States
(12) Patent Application Publication
(10) Pub. No.: US 2023/0404658 A1
(43) Pub. Date: Dec. 21, 2023

(54) A DEVICE FOR TREATMENT OF THE LEFT ATRIAL APPENDAGE

(71) Applicant: AURIGEN MEDICAL LIMITED, Galway (IE)

(72) Inventors: Tony O’HALLORAN, Co. Galway (IE); John THOMPSON, Dublin (IE); Conor ALLEN, Galway (IE)

(73) Assignee: AURIGEN MEDICAL LIMITED, Galway (IE)

(21) Appl. No.: 18/249,137

(22) PCT Filed: Oct. 15, 2021
(86) PCT No.: PCT/EP2021/078602

§ 371 Date: Apr. 14, 2023

(30) Foreign Application Priority Data
Oct. 17, 2020 (EP) 20202449.3

(51) Int. Cl. A61B 18/14; A61B 18/12; G16H 40/63

(52) U.S. Cl. CPC A61B 18/1492; A61B 2017/00172; G16H 40/63; A61B 18/1206

(57) ABSTRACT

A device (10) to occlude the left atrial appendage (1) of a heart of a subject comprises an implantable occlusion apparatus (30) configured for radial expansion upon deployment to fluidically occlude the left atrial appendage, an elongated catheter member (80) having a distal end attachable to the implantable occlusion apparatus for transluminal delivery of the implantable occlusion apparatus to the left atrial appendage, a tissue energising module (20) having a plurality of electrodes (26) disposed around a circumference of the implantable occlusion apparatus in which each electrode is configured to contact a wall of the left atrial appendage at a tissue focal point upon deployment of the implantable occlusion apparatus, and an electrical controller (40) including a pulsed field energy delivery generator operably attachable to an electrical power source (50) and the plurality of electrodes and configured to energise the electrodes in a pulsed field ablation modality. The electrical controller is configured to independently energise each of the plurality of electrodes to apply a non-uniform pulsed field ablation treatment circumferentially around the wall of the left atrial appendage.

### FIELD OF THE INVENTION

The present invention relates to treat the left atrial appendage. In particular the invention relates to a device to occlude the left atrial appendage electrically isolate the wall of the left atrial appendage by pulsed field electrical ablation.

### BACKGROUND TO THE INVENTION

Atrial fibrillation (AF) is a common cardiac rhythm disorder affecting an estimated 6 million patients in the United States alone. AF is the second leading cause of stroke in the United States and may account for nearly one-third of strokes in the elderly. As our population continues to age, this problem may become even more prevalent. In greater than 90% of cases where a blood clot (thrombus) is found in the AF patient, the clot develops in the left atrial appendage (LAA) of the heart. The irregular heart beat in AF causes blood to pool in the left atrial appendage, because clotting occurs when blood is stagnant, clots or thrombi may form in the LAA. These blood clots may dislodge from the left atrial appendage and may enter the cranial circulation causing a stroke, the coronary circulation causing a myocardial infarction, the peripheral circulation causing limb ischemia, as well as other vascular beds. The LAA is a muscular pouch of heart attached to the left atrium. Mechanical occlusion of the LAA may result in a reduction of the incidence of stroke in AF patients, and there is growing interest in both surgical and endovascular methods to remove isolate the LAA.

Anti-clotting drugs may be used to prevent strokes in patients diagnosed with AF. However, many people cannot take such drugs because of potential side effects. Drug therapy may also cause bleeding and may be difficult to control because determining dosage is challenging. Recent studies indicate that elimination of the LAA, through occlusion or closure, may prevent thrombi from forming in the LAA and thus may reduce the incidence of stroke in patients diagnosed with AF. As such, occlusion or closure of the LAA may significantly reduce the incidence of stroke in patients with atrial fibrillation and without the complications of drug therapy.

Historically, LAA's have sometimes been modified surgically, via suturing, clipping or excision to reduce the risk imposed by atrial fibrillation. In recent years, devices which may be delivered percutaneously into the left atrial appendage have been introduced. The basic function of these devices is to exclude the volume within the appendage with an implant which then allows blood within the appendage to safely thrombose and then to be gradually incorporated into cardiac tissue. This can leave a smooth, endothelialized surface where the appendage used to be.

New devices to percutaneously occlude the LAA have been developed for stroke prophylaxis and seem promising. These new devices include the use of a clip to clamp the LAA shut, the use of a snare to wall off the LAA, the use of an umbrella device to expand the LAA, the use of a device which may close the LAA but not electrically isolate it, and the use of a device which may fill the LAA without closing it. Data on the safety and efficacy of these devices must be considered over time. These new devices are early in clinical trials for human application and have several limitations. For instance, use of the clip to clamp the LAA shut may not get down to the base of the LAA, may leave a residual stump or leak, may result in a clot forming, and may require open surgery. Use of the snare may leave a residual stump or leak, may be less controlled, and may not be possible if adhesions are located around the heart. Use of the umbrella device may require the patient to be on blood thinners since it is made out of a foreign material and does not occlude and electrically isolate the LAA simultaneously. Use of a device which may close the LAA without electrical isolation, and use of a device which may electrically isolate the LAA without closing it are both incomplete solutions which may experience leakage, which may require blood thinners due to the use of synthetic materials, or which may experience other types of issues.

More recent devices proposed for occlusion of the LAA and prevention/treatment of atrial fibrillation and LAA-associated thrombotic events are described. WO2012/109297 describes an implantable device having an expandable LAA-occluding barrier and anchor configured for engagement of the ostium of the LAA, a pacing module for treatment of atrial fibrillation, and a sensor for detecting the electrical activity of the heart indicative of arrhythmia. WO2013/009872 describes a LAA-occluding device configured to inject a filler material into the LAA, and including a transponder unit configured to detect and relay to an external base station data electrical parameters of the LAA tissue. WO2016/202708 describes an implantable device having a LAA occluding body, electrodes configured to heat LAA tissue with a view to electrical isolation of the LAA, and sensors configured to determine heat or electrical activity of the LAA, which signals are used as feedback to control the heating of the tissue. While these devices are capable of occluding LAA's having regular openings, they are not suitable for use with LAA's having irregular shaped openings. In addition, while the devices may be operable to monitor and achieve electrical isolation of the LAA, in many cases they will not prevent subsequent atrial fibrillation events as electrical isolation achieved with the devices is reversible. A further problem with these devices is that the connector between the delivery catheter and the expandable barrier is disposed on the left atrial side of the barrier, and exposed to circulating blood, which can cause DRT (device-related thrombus) formation.

Stewart et al (Feasibility of Pulsed Field Intracardiac Ablation, Heart Rhythm, Vol. 16, No. 5, May 2019) describes a device for applying pulsed field ablation to the wall of the various heart structures including the pulmonary vein, right atrial appendage and left atrial appendage. The device comprises a 9-electrode circular array pulmonary vein ablation catheter powered by a PFA generator which delivers high voltage biphasic pulse trains to the multielectrode catheter through a cable that connects electrodes 1, 3, 5, 7 and 9 as one polarity and electrodes 2, 4, 6 and 8 as the opposite polarity.

US2020/008870 describes a device and system for ablating and occluding the left atrial appendage, comprising a radially expansible body having a plurality of electrodes arranged circumferentially on the body.

U.S. Pat. No. 6,416,505 describes a tissue ablation catheter and in particular focusses on solving a technical problem of providing ablating elements that can adapt to different shapes and sizes for different physiologic environments. The system includes a microcontroller configured to sequence successive power pulses to adjacent electrode regions so that the end of the duty cycle for the preceding pulse overlaps slightly with the beginning of the duty cycle for the next pulse and ensure an overlap in pulse duty cycles so that the source applies power continuously, with no periods of interruption caused by open circuits during pulse switching between successive electrode regions. Thus, the system is configured for distributing power to the multiple electrode regions to establish and maintain a uniform distribution of temperatures along the ablating element. The size and spacing of the electrode elements disclosed in D2 are well suited for creating continuous, long and curvilinear lesion patterns in tissue when ablation energy is applied simultaneously to adjacent emitting electrode elements.

It is an object of the invention to overcome at least one of the above-referenced problems.

### SUMMARY OF THE INVENTION

The Applicant has realised that the walls of some body lumens such as the left atrial appendage (LAA) have a non-uniform cross-section circumferentially, with some parts of greater thickness than others, and that the non-uniformity of the thickness of the wall varies from subject to subject. In addition, the electrical parameters of tissue including electrical impedance also varies circumferentially and between subjects. Therefore, a tissue ablation treatment such as pulsed field ablation (PFA) to deliver a uniform e-field circumferentially around the wall of the LAA (as provided by the systems of the prior art including US2020/008870) may lead to some parts being successfully ablated without damage to adjacent structures, and other (thinner) sections being over-treated with resultant damage to adjacent structures such as nerves. The present invention solves the above technical problem by providing a system that individually energises the elements or pairs of elements so as to enable some elements to be energised with greater ablative power than others to create a non-uniform tissue ablation circumferentially around the wall of the LAA, tuned to the specific anatomy of the subject. The device is configured to tune the energy delivery to the tissue ablative elements based on tissue electrical parameter data determined at a plurality of locations around the circumference of the body lumen. The device may incorporate a sensor to obtain tissue electrical parameter data or may be used in conjunction with a separate sensor to obtain the tissue electrical parameter data. In one embodiment, the controller/generator and ablative elements may be configured to operate in two separate modalities, a tissue parameter measurement modality and a tissue ablative treatment modality. This allows a tissue parameter such as electrical impedance to be determined at a plurality of locations around the circumference of the LAA (for example between electrodes or between an electrode and a ground pad), and a tissue parameter profile comprising the plurality of tissue parameter measurements to be generated. The device may include a processor configured to receive the tissue parameter measurements and compile the tissue parameter profile, and then modify the output characteristics of the controller/generator to independently energise the ablative elements during a treatment phase in a pattern synergistic with the tissue parameter profile. In the context of pulsed field ablation (PFA) of tissue, this means that a non-uniform e-field may be generated by the device, with PFA of greater power being delivered to certain locations around the circumference of the body lumen through the electrodes during a treatment phase.

In a first aspect, the invention provides a device to occlude a body lumen, for example the left atrial appendage of a heart, of a subject, comprising:
a implantable occlusion apparatus configured for radial expansion upon deployment to occlude the body lumen;
a tissue energising module having a plurality of tissue ablation elements (e.g. electrodes), usually disposed around a circumference of the implantable occlusion apparatus, in which each element is configured to contact a wall of the body lumen upon deployment of the implantable occlusion apparatus; and
an electrical controller coupled to an electrical power source and the plurality of elements and configured to energise the elements to ablate tissue of the wall of the body lumen.

The electrical controller is typically configured to independently energise each of the plurality of elements to apply a non-uniform tissue ablation treatment circumferentially around the wall of the left atrial appendage.

In any embodiment, the tissue ablation elements are electrodes.

In any embodiment, the controller comprises a pulsed field energy delivery generator operably connected to the electrodes and configured to energise the electrodes in a pulsed field ablation modality.

The device generally comprises an elongated catheter member having a distal end attachable to the implantable occlusion apparatus for transluminal delivery of the implantable occlusion apparatus to the left atrial appendage. The device may be configured for detachment of the catheter from the implantable occlusion apparatus in-vivo. This allows the device to be delivered and deployed, and then detachment of the implantable occlusion apparatus from the catheter and retraction of the catheter to leave the implantable occlusion apparatus in-situ in the LAA.

In any embodiment, the implantable occlusion apparatus is radially adjustable from a delivery configuration to a radially expanded deployed configuration.

In one embodiment, the device comprises a tissue parameter sensor configured to measure an electrical parameter of the tissue at one or a plurality of locations around the circumference of the wall of the body lumen. The tissue parameter sensor may comprise the electrical controller and the tissue energising module.

In another embodiment, the device is configured for use with a tissue parameter sensor that is separate to and configured to be used with the device of the invention. The device may be configured for the separate tissue parameter sensor to be delivered transluminally through a lumen of the catheter for deployment within the implantable occlusion apparatus and optional retraction after use.

In any embodiment, the device comprises a processor operably coupled to the electrical controller and configured to generate an electrical parameter profile of the wall of the body lumen comprising the electrical parameter measurements taken at the plurality of locations, for example a plurality of circumferential sections, and modify the output of the electrical controller so as to independently energise the electrodes (generally electrode pairs) in a pattern synergistic with the tissue parameter profile. The controller may be configured to take electrical parameter measurements using two electrodes, or a set of electrodes (e.g. four electrodes), especially when the electrical parameter is electrical impedance. The ability of the device to independently energise pairs of electrodes allows a non-uniform e-field to be generated around the circumference of the body lumen in which greater PFA power is applied at locations around the body where it is required as determined by the electrical parameter measurement at that location. For example, in the case of electrical impedance, the electrical impedance profile of the body identifies locations around the body where the electrical impedance of the tissue is highest, allowing PFA of lower power to be delivered at those locations through the electrodes at those locations.

In any embodiment, the electrical parameter profile comprises a plurality of electrical parameter measurements of circumferential sections of the wall of the body lumen. In any embodiment, the electrical parameter profile comprises a plurality of electrical parameter measurements of radial sections of the wall of the body lumen. In any embodiment, the electrical parameter profile comprises a plurality of electrical parameter measurements of circumferential and radial sections of the wall of the body lumen.

In any embodiment, the electrical parameter of the tissue is electrical impedance. Electrical impedance is generally employed to calculate the PFA energy to be applied to a section of the body lumen. An alternative electrical parameter of tissue is electrical activity (voltage) across a section of tissue. Electrical activity may be employed to determine the level of electrical occlusion of the body lumen due to the tissue treatment.

In any embodiment, the tissue parameter sensor is configured to measure an electrical parameter of the tissue at a plurality of focal points/sections around the circumference of the wall of the body lumen or radially across the wall of the body lumen.

In any embodiment, the tissue parameter sensor comprises the electrical controller and the tissue energising module, and in which the processor is configured to actuate the controller and tissue energising module in at least two separate modalities selected from a treatment (tissue ablation) modality and a sensing (tissue parameter measurement) modality. In any embodiment, the tissue ablation modality is configured to deliver a pulsed field ablation treatment at one or more of the plurality of locations around the circumference of the wall of the body lumen. In any embodiment, the tissue parameter measurement modality is configured to measure an electrical parameter of tissue at the plurality of locations around the circumference of the wall of the body lumen.

In any embodiment, the processor is configured to compare the electrical parameter measurement for a section of tissue with a reference electrical parameter value, calculate an energising power to be applied to electrodes at the section of tissue based on the comparison, and modify the output characteristics of the electrical controller to independently energise the electrodes according to the energising power calculated for the section of tissue. The electrode parameter measurements may be electrical impedance. In any embodiment, the electrical impedance of the section of tissue may be determined using two electrodes of the tissue energising module or four electrodes of the tissue energising module.

In any embodiment, the processor is configured to actuate the controller and tissue energising module in the tissue parameter measurement modality after a tissue ablation treatment.

In any embodiment, the processor is configured to actuate the controller and tissue energising module in the tissue parameter measurement modality to measure an electrical parameter circumferentially around a section of the wall of the left atrial appendage between two electrodes. The electrical controller generally includes a routing module configured to create an electrical circuit between any two electrodes of the tissue energising module and the controller. In this way, the controller can apply PFA to a circumferential section of the wall of the body lumen between any two electrodes. Generally during a treatment operation, the controller applies PFA ablation to a circumferential section of tissue between two adjacent electrodes. In any embodiment, the processor is configured to actuate the controller and tissue energising module in the tissue parameter measurement modality to measure an electrical parameter circumferentially around a section of the wall of the left atrial appendage using four electrodes. Methods of measuring electrical impedance of a section of tissue using a two-electrode circuit and a four electrode circuit are described in detail in Fraczek et al (Acto of Bioengineering and Biomechanics, Vol. 18, No. 1, 2016), especially FIGS. 3, 4 and 5 and the associated description. The use of a 4-electrode circuit has been found to reduce the incidence of electropolarisation which can affect the accuracy of electrical impedance measurements in tissue. A four-electrode embodiment is described in FIGS. 4 and 5 of Fraczek et al. The routing module of the controller may be configured to create two circuits using four electrodes of the tissue energising module as described in FIGS. 4 and 5 of Fraczek.

In any embodiment, the processor is configured to actuate the controller and tissue energising module in the tissue parameter measurement mode to measure an electrical parameter radially across the wall of the left atrial appendage. The device of the invention generally comprises an earth pad configured to be disposed on the surface of the subjects body (for example the chest of leg). The controller may be configured to measure an electrical parameter between the electrode and the earth pad and determine the electrical parameter radially across the wall of the body lumen.

In any embodiment, the tissue energising module comprises at least 4, 5, 6, 7, 8, 9, 10 electrodes disposed circumferentially around the wall of the implantable occlusion apparatus. In any embodiment, the electrodes are approximately equally spaced apart around a circumference of the implantable occlusion apparatus. In any embodiment, the tissue energising module comprises 8 electrodes disposed circumferentially around the wall of the implantable occlusion apparatus, and the processor and controller are preferably configured to measure electrical impedance across sections of the wall of the wall using two electrodes or four electrodes.

In any embodiment, a distal end of the catheter comprises a first electrical connection hub and the occlusion apparatus comprises a second electrical connection hub, wherein the electrical connection hubs are configured for detachable engagement to independently electrically couple each electrode with the controller through the catheter.

In any embodiment, each electrode is independently electrically connected to the controller by a connecting lead passing through a lumen of the catheter. In any embodiment, each lead has a proximal section at least partly disposed in the catheter and a distal section disposed on the implantable occlusion apparatus. The proximal section of each lead typically extends from the electrical controller (generally disposed outside the body of the subject) to a first electrical connecting hub disposed at a distal end of the catheter. The distal section of each lead extends from a second electrical connecting hub generally disposed on a proximal end of the implantable occlusion apparatus to an electrical hub. The first and second electrical connecting hubs are configured to provide electrical connection between the proximal and distal sections of each lead, when electrically connected, providing each electrode with its own electrical supply. The first and second electrical connecting hubs are typically configured for detachable electrical coupling. This allows the catheter to be detached from the implantable occlusion apparatus and the catheter to be retracted and removed from the body leaving the implantable occlusion apparatus in situ in the body. Typically, each lead is electrically insulated.

In another aspect, the invention provides a device to occlude a body lumen, for example the left atrial appendage of a heart, of a subject, comprising:
a implantable occlusion apparatus configured for radial expansion upon deployment to occlude the body lumen;
a tissue energising module having a plurality of electrodes disposed around a circumference of the implantable occlusion apparatus in which each electrode is configured to contact a wall of the body lumen upon deployment of the implantable occlusion apparatus;
an electrical controller including a pulsed field energy delivery generator operably coupled to an electrical power source and the plurality of electrodes and configured to independently energise the electrodes to apply a non-uniform pulsed field ablation treatment circumferentially around the wall of the left atrial appendage;
a tissue parameter sensor configured to measure an electrical parameter of the tissue at a plurality of locations around the circumference of the wall of the body lumen; and
a processor operably coupled to the electrical controller and configured to generate an electrical parameter profile of the body lumen comprising electrical parameter measurements taken at the plurality of locations, and modify the output of the electrical controller so as to independently energise the electrodes in a pattern synergistic with the electrical parameter profile.

The plurality of locations may include circumferential sections of the wall of the body lumen, radial sections of the wall of the body lumen, or both.

In any embodiment, the tissue parameter sensor comprises the electrical controller and the tissue energising module, and in which the processor is configured to actuate the controller and tissue energising module in at least two separate modalities selected from a treatment (tissue ablation) modality and a sensing (tissue parameter measurement) modality.

In any embodiment, the tissue ablation modality is configured to deliver a pulsed field ablation treatment at one or more of the plurality of locations around the circumference of the wall of the body lumen. Generally, each location is a circumferential section of wall of the body lumen defined by an electrode pair.

In any embodiment, the controller is configured to deliver at least one train of PFA energy to an electrode.

In any embodiment, the controller is configured to deliver a train of energy of at least 20 pulses to an electrode.

In any embodiment, the controller is configured to deliver at least one train of energy comprising an inter-phase delay of between 0 μs and 100 μs.

In any embodiment, the controller is configured to deliver a train of energy comprising an inter-pulse delay of at 1 to 100 μs and typically at least 5 μs.

In any embodiment, the controller is configured to deliver a train of energy comprising a pulse width of 100 ns-100 μs.

In any embodiment, the controller is configured to deliver at least one train of PFA energy having a voltage amplitude between 100V and 5000V.

In any embodiment, the controller is configured to deliver pulses in monophasic or biphasic form.

In any embodiment, the electrodes have a monopolar or bipolar configuration. Thus, in a sensing mode, electrical impedance may be measured between two electrodes forming part of the occlusion apparatus, or between one electrode on the occlusion apparatus and a reference electrode (ground pad).

In any embodiment, the device is configured to:
deliver by the controller and electrodes an electrical (e.g. pulsed field ablation) treatment at the section of the wall of the body lumen,
determine by the controller and electrodes an electrical parameter measurement of the tissue at the treated section; and
correlate by the processor an output indication of electrical isolation of the section of the wall of the body lumen based on the electrical parameter measurements.

The processor may be configured to receive as an input the electrical parameter measurement, compare the measurement with one or more reference value for the electrical parameter, and calculate an output indication of electrical isolation of the section of the wall based on the comparison. Typically, the reference values comprise values obtained with a training set of electrical parameter values.

In any embodiment, the device is configured to:
determine by the controller and electrodes a first electrical parameter measurement (such as a first electrical impedance or electrical activity value) of tissue at a section of the wall of the body lumen,
deliver by the controller and electrodes an electrical (e.g. pulsed field ablation) treatment at the section of the wall of the body lumen,
determine by the controller and electrodes a second electrical parameter measurement of the tissue at the treated section; and
calculate by the processor an output indication of electrical isolation of the section of the wall of the body lumen based on a comparison of the first and second electrical parameter measurements.

In one embodiment, the processor is configured to generate a an output indicative of electrical isolation of the section of the wall when a reduction in the electrical parameter values before and after treatment is calculated. In one embodiment, a reduction of at least 10%, 20%, 30%, 40%, 50%, 60%, 70%, 80% or 90% is indicative of electrical isolation of the section of tissue. In one embodiment, the processor is configured to display the output indication on an interface.

In any embodiment, the generator is configured to deliver (or the method of the invention comprises delivering) at least one pulse train of energy (typically at a predetermined frequency) to the tissue of the body lumen, each pulse train of energy including:
at least 60 pulses,
an inter-phase delay between 0 μs and 5 μs,
an inter-pulse delay of 2 to 500 μs,
a pulse width of 1 to 15 μs, and
a voltage of 500 to 2500 V.

In any embodiment, the radially expansible body is configured to fluidically occlude the body lumen when in an expanded configuration.

In any embodiment, one or more or all of the electrodes comprise an energy delivery surface comprises a central electrically conducting region and a peripheral electrically insulating region.

In any embodiment, the implantable occlusion apparatus is a radially expansible body comprising 4 to 16 electrodes circumferentially spaced around the external body lumen facing surface of the radially expansible body.

In any embodiment, the radially expansible body comprises 6 to 10 electrodes circumferentially spaced around the external body lumen facing surface of the radially expansible body.

In any embodiment, adjacent electrodes are spaced apart by about 3 to about 14 mm centre to centre.

In any embodiment, adjacent electrodes are spaced apart by about 6 to about 14 mm centre to centre.

In any embodiment, one or more of the electrodes is planar.

In any embodiment, one or more of the planar electrodes has an oblong profile and an electrode longitudinal axis, wherein the electrode is mounted to the radially expansible body such that the electrode longitudinal axis is parallel to a longitudinal axis of the radially expansible body.

In any embodiment, one or more of the electrodes has a width of about 1.5 mm to about 5 mm.

In any embodiment, one or more of the electrodes has a width of about 1.5 mm to about 3 mm.

In any embodiment, one or more of the electrodes has a length of about 2 mm to about 8 mm or about 4 to about 8 mm.

In any embodiment, one or more of the electrodes has a length of about 5 mm to about 7 mm.

In any embodiment, the generator is configured to deliver at least one pulse train of energy with inter-pulse delay is about 2 to about 400 μs.

In any embodiment, the generator is configured to deliver at least one pulse train of energy with inter-pulse delay is about 2 to about 250 μs.

In any embodiment, the generator is configured to deliver at least one pulse train of energy with inter-pulse delay is about 200 to about 400 μs.

In any embodiment, the pulse width is 1 to 5 μs.

In any embodiment, each pulse train of energy includes:
at least 60 pulses;
an inter-phase delay between 0 μs and 5 μs;
an inter-pulse delay of 200 to 500 μs;
a pulse width of 1 to 5 μs, and
a voltage of 750 to 2500 V;
In any embodiment, the pulse train of energy includes biphasic pulses.

In any embodiment, the generator is configured to deliver a plurality of pulse trains with a pulse train delay of 50-800 μs.

In any embodiment, the generator is configured to deliver a sequence of pulse trains comprising 100 to 300 pulse trains.

In any embodiment, the generator is configured to deliver a pulse train of energy with a pulse on-time of 30 to 90 ms.

In any embodiment, the radially expansible body is detachably attachable to the catheter.

In any embodiment, the generator is configured to deliver AC or DC current.

In any embodiment, the plurality of electrodes comprise one or more electrode pairs, and the system is configured to deliver the pulse field ablation energy across the spaced apart electrodes of at least one electrode pair.

In any embodiment, the plurality of electrodes comprise three spaced apart electrodes including a central electrode, a first electrode disposed on one side of the central electrode and a second electrode disposed on an opposite side of the central electrode, and the system is configured to deliver the pulse field ablation energy from the first electrode to the central electrode and from the second electrode to the central electrode.

In another aspect, the invention provides a method of treating a body lumen comprising the steps of:
deploying a implantable occlusion apparatus within the body lumen in which the implantable occlusion apparatus has a plurality of electrodes disposed circumferentially around the implantable occlusion apparatus configured for contacting a wall of the body lumen upon deployment of the implantable occlusion apparatus;
measuring an electrical parameter of the body lumen tissue at a plurality of spaced-apart circumferential locations around the wall of the body lumen;
compiling the electrical parameter measurements into a body lumen specific tissue parameter profile; and
independently energising the electrodes to apply pulsed field ablation (PFA) circumferentially around the wall of the LAA in a pattern synergistic with the tissue parameter profile.

In any embodiment, the electrical parameter measured in electrical impedance of the tissue. In any embodiment, the electrical parameter is measured across a circumferential section of the wall of the body lumen defined by two electrodes. In any embodiment, the electrical parameter is measured across a radial section of the wall of the body lumen.

In any embodiment, the implantable occlusion apparatus is configured to fluidically occlude the body lumen upon deployment (e.g. it blocks the lumen and prevents fluid passage past the implantable occlusion apparatus).

In any embodiment, the body lumen is part of the blood circulatory system, for example a blood vessel, for example a vein or artery. In any embodiment, the body lumen is a left atrial appendage. Other body lumen include lumens forming part of the respiratory, renal or hepatic systems.

In any embodiment, the method comprises measuring an electrical parameter of the body lumen tissue at least at 3, 4, 5, 6, 7, 8, 9 or 10 spaced-apart circumferential locations around the wall of the body lumen.

In any embodiment, the method employs an electrical controller including a pulsed field energy delivery generator operably coupled to an electrical power source and the plurality of electrodes and configured to energise the electrodes in a pulsed field ablation modality.

In any embodiment, the method employ a processor operably coupled to the electrical controller and configured to receive electrical parameter measurements, generate a body lumen specific electrical parameter profile, and modify the output characteristics of the electrical controller to independently energise the electrodes to apply pulsed field ablation (PFA) circumferentially around the wall of the LAA in a pattern synergistic with the tissue parameter profile.

In any embodiment, the method comprises the steps of applying pulsed field ablation to the wall of the body lumen, determining a tissue parameter profile of the body lumen, and optionally applying a further pulsed field ablation to the wall of the body lumen.

In any embodiment, the method employs an elongated catheter having a distal end coupled to the implantable occlusion apparatus, and a step of transluminal delivery of the implantable occlusion apparatus to the body lumen. In any embodiment, the implantable occlusion apparatus is radially adjustable from a delivery configuration to a radially expanded deployed configuration, and the method comprises a step of deployment of the implantable occlusion apparatus in the body lumen.

In any embodiment, the method comprises a step of detaching the catheter from the implantable occlusion apparatus after PFA treatment of the tissue, and transluminal retraction of the catheter leaving the implantable occlusion apparatus in-situ.

In any embodiment, the method comprises delivering at least one train of PFA energy to an electrode.

In any embodiment, the method comprises delivering at least one train of energy of at least 20 pulses to an electrode.

In any embodiment, the at least one train of energy comprises an inter-phase delay of between 0 μs and 100 μs.

In any embodiment, the train of energy comprises an inter-pulse delay of 1-100 μs and typically at least 5 μs.

In any embodiment, the train of energy comprises a pulse width of 100 ns-100 μs.

In any embodiment, the at least one train of PFA energy has a voltage amplitude between 100V and 5000V.

In any embodiment, the pulse delivery in selected from monophasic or biphasic

In any embodiment, the electrodes have a monopolar or bipolar configuration.

In any embodiment, the method comprises the steps of:
delivering by the controller and electrodes an electrical (e.g. pulsed field ablation) treatment at the section of the wall of the body lumen,
determining by the controller and electrodes an electrical parameter measurement of the tissue at the treated section; and
correlating by the processor an output indication of electrical isolation of the section of the wall of the body lumen based on the electrical parameter measurements.

The process may comprise receiving as an input the electrical parameter measurement, comparing the measurement with one or more reference value for the electrical parameter, and calculating an output indication of electrical isolation of the section of the wall based on the comparison. Typically, the reference values comprise values obtained with a training set of electrical parameter values.

In any embodiment, the method comprises:
determining by the controller and electrodes a first electrical parameter measurement (such as a first electrical impedance or electrical activity value) of tissue at a section of the wall of the body lumen,
delivering by the controller and electrodes an electrical (e.g. pulsed field ablation) treatment at the section of the wall of the body lumen,
determining by the controller and electrodes a second electrical parameter measurement of the tissue at the treated section; and
calculating by the processor an output indication of electrical isolation of the section of the wall of the body lumen based on a comparison of the first and second electrical parameter measurements.

In any embodiment, the method comprises generate an output indicative of electrical isolation of the section of the wall when a reduction in the electrical parameter values before and after treatment is calculated. In one embodiment, a reduction of at least 10%, 20%, 30%, 40%, 50%, 60%, 70%, 80% or 90% is indicative of electrical isolation of the section of tissue. In one embodiment, the processor is configured to display the output indication on an interface.

In any embodiment, the method comprises re-calibration of the electrical treatment based on the output indication of electrical isolation calculated by the processor.

The invention also relates to a method of electrically occluding a body lumen using a device and method of the invention. The invention also relates to a method of treating atrial fibrillation in a subject by electrical occlusion of heart structure in the subject using a device and method of the invention.

Other aspects and preferred embodiments of the invention are defined and described in the other claims set out below.

### BRIEF DESCRIPTION OF THE FIGURES

FIG. 1 is a sectional view of left atrial appendage (LAA) illustrating the non-uniformity of the thickness of the wall of the LAA.

FIG. 2 is a sectional view of left atrial appendage illustrating the non-uniformity of the electrical impedance of the tissue along circumferential sections.

FIG. 3 illustrates the device of the invention including delivery catheter, implantable occlusion apparatus, electrical controller and processor.

FIG. 4 illustrates in detail a deployable device of the invention including a circumferential array of electrodes and a distal end of a catheter connected to a recessed connecting hub of the occlusion apparatus.

FIG. 5 illustrates a non-uniform e-field applied by a device of the invention to the wall of the LAA

FIG. 6 illustrates an arrangement of electrodes for measuring electrical impedance along a circumferential section of the wall of the LAA.

FIG. 7 illustrates an arrangement of electrodes for measuring electrical impedance radially across a section of the wall of the LAA.

FIG. 8 is a block diagram illustrating the use of the device of the invention to occlude the LAA by pulsed field ablation.

FIG. 9 is an illustration of an occlusion apparatus having an array of electrodes deployed in the LAA and the use of the electrodes to determine the electrical impedance of circumferential sections of the LAA.

FIG. 10 is an illustration of an occlusion apparatus having an array of electrodes deployed in the LAA and the use of the electrodes to determine the electrical impedance radially across the wall of the LAA at eight positions around the wall of the LAA.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of a PFA treatment to a subject) which cures, ameliorates or lessens the symptoms of a disease or removes (or lessens the impact of) its cause(s). In this case, the term is used synonymously with the term “therapy”.

Additionally, the terms “treatment” or “treating” refers to an intervention (e.g. the administration of a PFA treatment to a subject) which prevents or delays the onset or progression of a disease or reduces (or eradicates) its incidence within a treated population. In this case, the term treatment is used synonymously with the term “prophylaxis”.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Implantable occlusion apparatus” means an apparatus configured for implantation in a body lumen, especially implantation in the heart at least partially or fully within the left atrial appendage, and upon actuation to at least partially or fully fluidically occlude the body lumen. The occlusion apparatus is typically detachably connected to a delivery catheter which delivers the occlusion apparatus to the target site, and typically remains attached during occlusion, sensing and energy delivery treatments and in one embodiment is generally detached after the energy delivery treatment and removed from the body leaving the occlusion apparatus implanted in the body lumen. Occlusion may be complete occlusion (closing) of the body lumen or partial occlusion (narrowing of the body lumen or near complete occlusion). The occlusion apparatus typically comprises a body that is expansible from a contracted delivery configuration to an expanded deployed configuration. The body may take many forms, for example a wireframe structure formed from a braided or meshed material. Examples of expandable wireframe structures suitable for transluminal delivery are known in the literature and described in, for example, WO01/87168, U.S. Pat. No. 6,652,548, US2004/219028, U.S. Pat. Nos. 6,454,775, 4,909,789, 5,573,530, WO2013/109756. Other forms of bodies suitable for use with the present invention include plate or saucer shaped scaffolds, or stents. In one embodiment, the body is formed from a metal, for example a shape-memory metal such as nitinol. The body may have any shape suitable for the purpose of the invention, for example cylindrical, discoid or spheroid. In one preferred embodiment, the apparatus comprises a cylindrical body, for example a cylindrical cage body. In one embodiment, the body comprises a tissue energising module. In one embodiment, the ablation device comprises an array of electrodes, typically a circumferential array. In one embodiment, the array of electrodes are configured to deliver pulsed field ablation to the tissue. In one embodiment, a distal face of the radially expansible body comprises a covering configured to promote epithelial cell proliferation. In one embodiment, the body comprises a stepped radial force stiffness profile from distal to proximal device. In one embodiment, the body comprises a metal mesh cage scaffold. In one embodiment, a coupling between the body and the catheter member is located distally to the left atrial facing side of the body. In one embodiment, the body in a deployed configuration has a radial diameter at least 10% greater than the radial diameter of the left atrial appendage at a point of deployment. In one embodiment, the furthermost distal part is configured to be atraumatic to cardiac tissue. In one embodiment, the body comprises a braided mesh scaffold that in one embodiment is conducive to collagen infiltration on thermal energy delivery to promote increased anti migration resistance. Examples of an implantable occlusion apparatus for use in a body lumen especially the LAA are described in WO2018/185256, WO2018/185255 and WO2020/074738.

“Body lumen” means a cavity in the body, and may be an elongated cavity such as a vessel (i.e. an artery, vein, lymph vessel, urethra, ureter, sinus, auditory canal, nasal cavity, bronchus) or an annular space in the heart such as the left atrial appendage, left ventricular outflow tract, the aortic valve, the mitral valve, mitral valve continuity, or heart valve or valve opening.

“Detachably attached” means that the device is configured such that the occlusion apparatus is attached to the elongated delivery catheter during delivery and can be released after deployment and treatment whereby the occlusion apparatus is implanted in the heart and the elongated delivery catheter can be withdrawn leaving the occlusion apparatus in-situ. Typically, the device includes a control mechanism for remotely detaching the occlusion apparatus or radially expansible element from the elongated catheter member. Typically, an actuation switch for the control mechanism is disposed on the control handle.

“Transluminal delivery” means delivery of the occlusion apparatus to a target site (for example the heart) heart through a body lumen, for example delivery through an artery or vein. In one embodiment, the device of the invention is advanced through an artery or vein to deliver the occlusion apparatus to the left atrium of the heart and at least partially in the LAA. In one embodiment, the device is delivered such that the distal part is disposed within the LAA and the proximal part is disposed in the left atrium just outside the LAA. In one embodiment, the device is delivered such that the distal part is disposed within the LAA and the proximal part is disposed in the left atrium abutting a mouth of the LAA. In one embodiment, the device is delivered such that both the distal and proximal parts are disposed within the LAA.

“Cover”: Typically, the implantable occlusion apparatus has a proximal cover which is impermeable to blood and that may include a re-closable aperture, for example an overlapping flap of material. The re-closable aperture may be configured to allow a distal end of the catheter through the aperture while preventing blood flow through the aperture. The occlusion apparatus may include a connecting hub distal of the cover, and configured for coupling with a distal end of the catheter. The cover may be configured to act as a scaffold for in-vivo endothelialisation. The cover may be formed from a woven mesh material.

“Covering/cover configured to act as a scaffold for in-vivo endothelialisation” means a material that is use promotes epithelialisation of the distal or proximal body. In one embodiment, the covering is a membrane that comprises agents that promote epithelial cell proliferation. Examples include growth factors such as fibroblast growth factor, transforming growth factor, epidermal growth factor and platelet derived growth factor, cells such as endothelial cells or endothelial progenitor cells, and biological material such as tissue or tissue components. Examples of tissue components include endothelial tissue, extracellular matrix, sub-mucosa, dura mater, pericardium, endocardium, serosa, peritoneum, and basement membrane tissue. In one embodiment, the covering is porous. In one embodiment, the covering is a biocompatible scaffold formed from biological material. In one embodiment, the covering is a porous scaffold formed from a biological material such as collagen. In one embodiment, the covering is a lyophilised scaffold.

“Tissue energising module” as used herein refers to an array of electrodes disposed on the implantable occlusion apparatus (e.g. a radially expansible body) configured for electrical coupling with the electrical controller. The electrodes are generally individually coupled with the controller to allow electrode specific energising of the electrode. They array of electrodes is generally arranged on the implantable occlusion apparatus in a circumferential arrangement and configured to contact the wall of the body lumen in a circumferential pattern when the apparatus is deployed. The electrodes are configured to deliver energy, generally PFA, circumferentially around the wall of the body lumen. The electrodes may also function as sensors to detect an electrical parameter of the tissue of the wall of the body lumen, for example electrical impedance or electrical activity (voltage). The electrodes may be configured to measure an electrical parameter radially across the wall of the body lumen, or circumferentially along a section of the circumference of the wall of the body lumen. Generally, measuring an electrical parameter such as electrical impedance radially across the wall of the body lumen employs an electrode of the array of electrodes and an earth or ground pad placed on the patient's body, often the leg. Measuring an electrical parameter such as electrical impedance circumferentially along a section of the body lumen employs two electrodes where one electrode functions as an energising electrode and the other functions as a detecting electrode. The electrical parameter such as electrical impedance may be measured at one frequency or over a range of frequencies.

“Independently energise” as applied to the device means that at least a plurality of the electrodes may be energised differently to apply a non-uniform energy (e.g. PFA) treatment to the body lumen. Thus, the present invention solves the technical problem of the prior art systems by individually energising the elements or pairs of elements so as to enable some elements to be energised with greater ablative power than others to create a non-uniform tissue ablation circumferentially around the wall of the LAA, tuned to the specific anatomy of the subject. The device is configured to tune the energy delivery to the tissue ablative elements based on tissue electrical parameter data determined at a plurality of locations around the circumference of the body lumen. The power of the treatment of tissue may be varied, for example in the case of PFA by varying the applied current or applied voltage. The device of the invention may be configured to energise the electrodes during a PFA treatment simultaneously or at different time points. The or each electrode may be energised at one frequency, or energised at different frequencies.

“Electrical parameter profile” describes a plurality of electrical parameter measurements taken at different locations along the circumference of the wall of the body lumen. It represents a body lumen specific fingerprint comprising electrical parameter measurements and can be used to tune the energy (e.g. PFA) treatment to avoid damage to tissue during energy treatment. The electrical parameter is generally electrical impedance, although other electrical parameters such as electrical activity may be employed. The electrical parameter profile is determined at a part of the wall of the body lumen where the electrodes are disposed. For example, where the device is configured to have electrodes positioned at 0°, 90°, 180° and 270° around the circumference of the wall of the body lumen, the electrical parameter profile may be a compilation of the values of electrical impedance at these across the four sections defined by these electrodes. A processor may be configured to receive the electrical impedance values, compile an electrical parameter profile, and generate output characteristics for the controller based on the electrical parameter profile. Generating the electrical parameter profile may comprise comparing each electrical parameter measurement with a reference electrical parameter measurement. In cases where the tissue electrical impedance measurements are not the same, the processor will be configured to modify the output characteristics of the controller to energise the electrodes unevenly and generate a non-uniform e-field around the wall of the body lumen, where areas of tissue with higher impedance will be treated by energy (e.g. PFA) of lower power and areas of tissue with lower impedance will be treated by energy (e.g. PFA) of higher power. This help avoid over-treatment of areas of tissue of high electrical impedance and helps reduce or avoids damage to adjacent structures and nerves due to over-treatment.

“Independently energise the electrodes in a pattern synergistic with the tissue parameter profile” means that the device is configured to individually energise the electrodes or pairs of electrodes so as to enable some electrodes to be energised with greater ablative power than others to create a non-uniform tissue ablation circumferentially around the wall of the LAA, tuned to the specific anatomy of the subject. The device is configured to tune the energy delivery to the tissue ablative electrodes based on tissue electrical parameter data determined at a plurality of locations around the circumference of the body lumen. Thus, the device can energise electrodes in the array so as to generate a uniform or non-uniform e-field depending on the electrical parameter profile of the body lumen. Thus, where the electrical parameter measurements are equal around the circumference of the wall of the LAA, the device is configured to energise the electrodes around the wall of the body lumen with equal or similar energy. Likewise, where the electrical parameter measurements are unequal around the circumference of the wall of the LAA, the device is configured to independently energise the electrodes around the wall of the body lumen unequally, to generate a non-uniform e-field which is higher in some areas (e.g. areas of low tissue impedance) and lower in other areas (e.g. areas of high tissue impedance). Generally a processor is employed to independently energise the electrodes in a pattern synergistic with the tissue parameter profile, and the processor may be configured to receive electrical parameter measurements, generate a body lumen specific electrical parameter profile, and energise the electrodes in a pattern synergistic with the tissue parameter profile.

“Electrical controller” refers to a pulsed field energy delivery generator that comprises or can be operably coupled to an electrical power source and is operatively coupled to the plurality of electrodes and configured to energise the electrodes, typically in a pulsed field ablation modality. In one preferred aspect, the controller comprises a signal generator configured for generating a pulse waveform. In any embodiment, the signal generator is configured to deliver at least one train of PFA energy to an electrode. In any embodiment, the signal generator is configured to deliver a train of energy of at least 20 pulses to an electrode. In any embodiment, the signal generator is configured to deliver at least one train of energy comprising an inter-phase delay of between 0 μs and 100 μs. In any embodiment, the signal generator is configured to deliver a train of energy comprising an inter-pulse delay of 1 to 100 μs, and typically at least 5 μs. In any embodiment, the signal generator is configured to deliver a train of energy comprising a pulse width of 100 ns-100 μs. In any embodiment, the signal generator is configured to deliver at least one train of PFA energy having a voltage amplitude between 100V and 5000V. In any embodiment, the signal generator is configured to deliver pulses in monophasic or biphasic form. The electrical controller is operably coupled to some or all of the electrodes (or electrode pairs) in the array in a manner allowing electrode pairs to be energised independently. The electrical controller may a comprise a plurality of electrode channels, and optionally a routing channel. Such an electrical controller is described in US2020230403. Electrical controllers for generating pulsed field ablative energy are described in EP3399933, US2020046423, WO2019157359 and US2020139114. Fraczek et al. describes the use of two electrodes or four electrodes to measure electrical impedance in tissue.

“Atrial fibrillation” or “AF” is a common cardiac rhythm disorder affecting an estimated 6 million patients in the United States alone. AF is the second leading cause of stroke in the United States and may account for nearly one-third of strokes in the elderly. In greater than 90% of cases where a blood clot (thrombus) is found in the AF patient, the clot develops in the left atrial appendage (LAA) of the heart. The irregular heartbeat in AF causes blood to pool in the left atrial appendage, because clotting occurs when blood is stagnant, clots or thrombi may form in the LAA. These blood clots may dislodge from the left atrial appendage and may enter the cranial circulation causing a stroke, the coronary circulation causing a myocardial infarction, the peripheral circulation causing limb ischemia, as well as other vascular beds. The term includes all forms of atrial fibrillation, including paroxysmal (intermittent) AF and persistent and longstanding persistent AF (PLPAF).

“Ischaemic event” refers to a restriction in blood supply to a body organ or tissue, resulting in a shortage of oxygen and glucose supply to the affected organ or tissue. The term includes stroke, a blockage of blood supply to a part of the brain caused by a blood clot blocking the blood supply to the brain and the resultant damage to the affected part of the brain, and transient ischaemic events (TIA's), also known as “mini-strokes”, which are similar to strokes but are transient in nature and generally do not cause lasting damage to the brain. When the restriction in blood supply occurs in the coronary arteries, the ischaemic event is known as a myocardial infarction (MI) or heart attack.

“Electrical impedance” refers to the opposition that a volume of tissue presents to an alternating electrical current when a sinusoidal voltage is applied across the volume of tissue. The actual definition of impedance is by Ohm's law, is the ratio of the complex voltage to the complex current

$$\vec{Z} = \frac{\vec{V}}{\vec{I}} \qquad ( 2.29 )$$

The active method of measurement (which is most likely method of measurement for the invention) are defined as the measure of the ratio of complex voltage to complex current following the above condition stated by Ohm's law.

$$\vec{Z} = \vert Z \vert < ∅ = \frac{\vert V \vert < {∅}_{v}}{\vert I \vert < {∅}_{i}} = \frac{\vert V \vert}{\vert I \vert} < {∅}_{v} - < {∅}_{i} \qquad ( 2.3 )$$

Where |Z|, |V|, |I| are the absolute magnitude values of the impedance, voltage and the current and the phase angles are represented by Ø_{z}, Øᵥ, Øᵢ respectively. The different kinds of active instruments are:

An impedance analyser chip or module may be attached to the controller/generator, this module will directly measure the magnitude of voltage and current and associated phase angles and then calculate angle and phase of the impedance. Other methods of measuring electrical impedance in tissue are described in, for example, Fraczek et al. and Sharp et al. (Saudi Journal of Anaesthesia, 2017, 11(1):15) and Kwon et al. (Scientific Reports 9, Article Number 3145 (2109)).

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

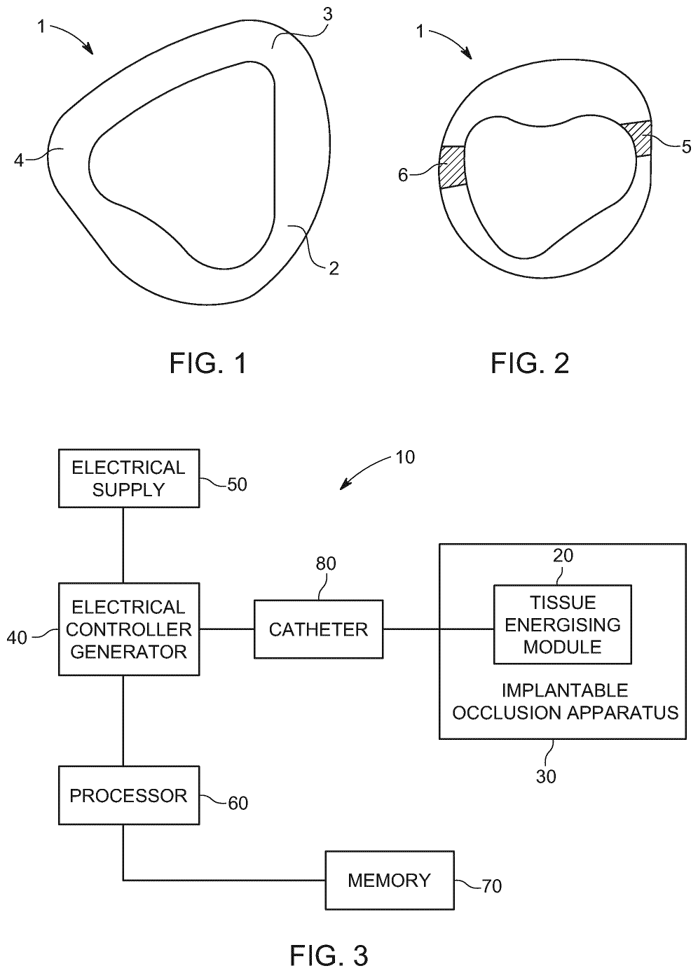

Referring to the drawing, and initially to FIG. 1, these is illustrated a left atrial appendage (LAA) 1 of a human heart shown in sectional view. The wall of LAA has a non-uniform cross section with wall sections 2, 3 and 4 of different thickness.

Referring to FIG. 2, there is illustrated another LAA 1 shown in sectional view, in which the LAA has circumferential sections 5 and 6 having different electrical impedance characteristics despite having the same wall thickness.

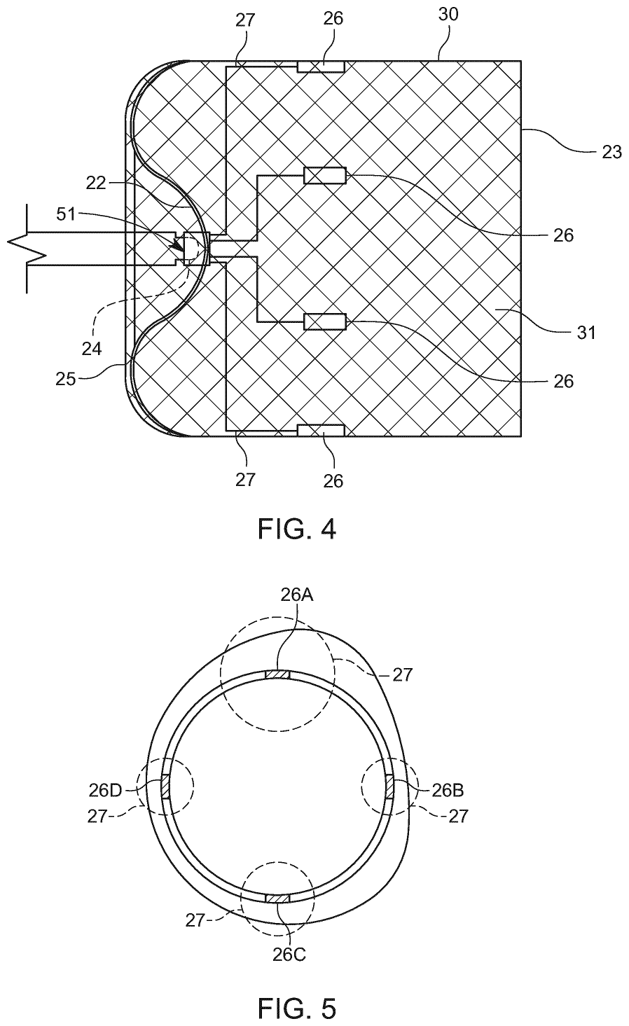

Referring to FIGS. 3 and 4, a device according to the invention, indicated generally by the reference numeral 10, is illustrated. Referring initially to FIG. 3, the device comprises a tissue energising module 20 disposed on an implantable occlusion apparatus 30, an electrical controller/generator 40 electrically coupled to a power source 50, a processor 60 and memory module 70. The tissue energising module 20 is electrically coupled to the controller 40 by insulated leads (not shown) provided in the lumen of an elongated catheter 80. The processor 60 is electrically coupled to the controller/generator 40 and configured to receive tissue parameter data from the controller and control the output characteristics of the controller when it energises the electrodes in a pulsed field ablation modality.

Referring to FIG. 4, the implantable occlusion apparatus 30 and tissue energising module 20 are described in more detail. The implantable occlusion apparatus 30 comprises a cylindrical mesh cage 31 with a recessed proximal end 22 and an open distal end 23. A connection hub 24 is provided on the proximal end, distal of a blood-impermeable cover membrane 25 having an aperture (not shown). An array of six electrodes 26 (four are illustrated in the drawing) are provided circumferentially around the wall of the occlusion apparatus at equally spaced apart locations. Each electrode 26 is connected to the connecting hub with a dedicated insulated lead 27. The distal end of the catheter has a connecting hub 51 configured for detachable engagement and electrical coupling with the connecting hub 24. The catheter has six insulated leads (not shown) that electrically connect the contacts of the connecting hub with the controller. When the hubs 24 and 51 are electrically connected, each electrode is electrically connected to the controller with a dedicated electrical lead, allowing each electrode to be independently energised.

The processor 60 controls the operation of the controller/generator 40 and can modify the output characteristics of the controller to operate in a treatment modality and in a sensing modality. In the sensing modality, the electrical impedance of tissue at a plurality of locations around the circumference of the LAA can be determined. For example, referring to FIG. 5, the electrical impedance of a circumferential section of the wall of the LAA defined between electrodes 26A and 26B can be measured by modifying the controller to create a circuit between these electrodes and the controller, energising one of the electrodes, and detecting voltage and current across the electrodes at a given frequency (or optionally at a plurality of different frequencies). In this manner, the array of electrodes can be employed in a sensing modality to determine the electrical impedance of the tissue of the LAA around the full circumference of the wall of the LAA, and the processor can compile the electrical impedance values into the electrical impedance profile which is specific to the LAA of the subject.

In a treatment modality, the processor is configured to calculate the pulsed field ablation energy to be applied to an electrode pair (e.g. electrodes 26A and 26B) based on the electrical impedance value for the section of tissue defined by the electrode pair. Thus, for a section of the wall of the LAA where the electrical impedance is determined to be high, the electrode pair bordering the section of the wall will be energised with low PFA power, and for a section of the wall of the LAA where the electrical impedance is determined to be low, the electrode pair bordering the section of the wall will be energised with higher PFA power. In this way, the processor controls the output characteristics of the controller so as to independently energise the electrodes in a pattern synergistic with the tissue parameter profile. In cases where the electrical impedance of the tissue around the wall of the LAA is non-uniform, this results in a non-uniform circumferential e-field being created around the wall of the LAA, illustrated in FIG. 5, where the e-fields 27 generated at the electrodes 26A to D are different in size providing a composite non-uniform e-field having areas of larger e-field located where the electrical impedance of the tissue is low and smaller e-fields at locations where the electrical impedance of the tissue is higher. This helps avoid over treating tissue and consequent damage to adjacent structures such as nerves and blood vessels.

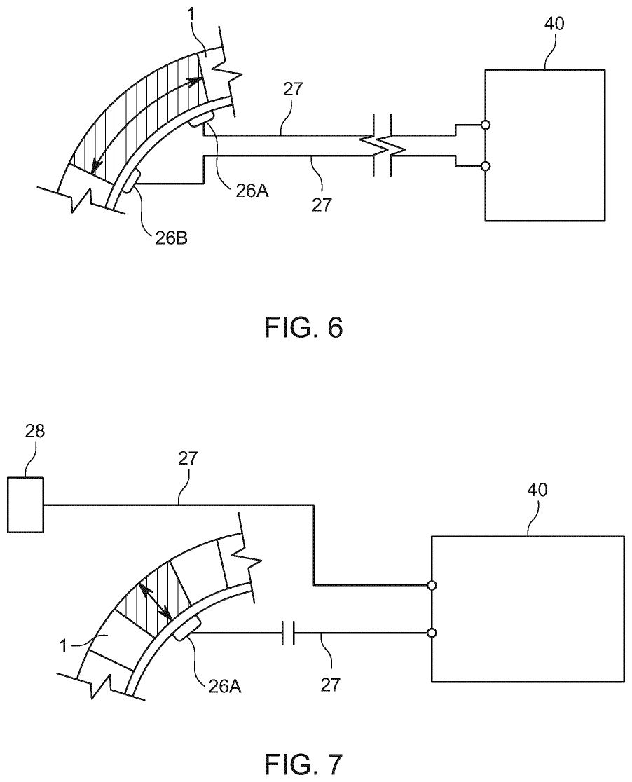

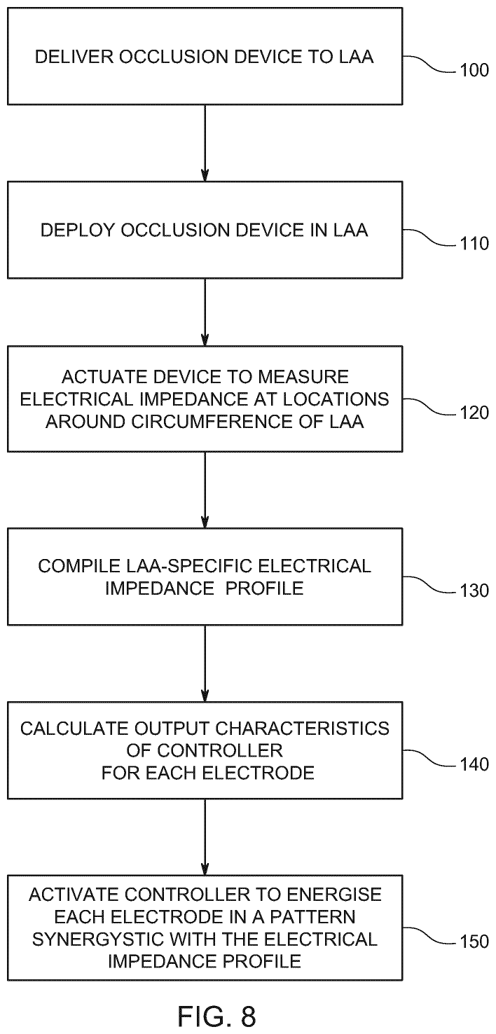

Referring to FIG. 6, a part of the LAA 1 and tissue energising module comprising electrodes 26A and 26B is shown along with leads 27 electrically coupling the electrodes with the controller 40. In a sensing mode, the controller energises one electrode to pass a current through the tissue for detection by the second electrodes and the current and voltage between the electrodes is determined and use to calculate the electrical impedance circumferentially across the shaded section of tissue.

Referring to FIG. 7, a part of the LAA 1 and tissue energising module comprising electrodes 26A is shown along with lead 27 electrically coupling the electrode with the controller 40. A ground pad 28 placed remotely to the LAA on the leg of the patient and electrically coupled to the controller 40 is also illustrated. In an alternative sensing mode, the controller energises the electrode to pass a current radially across the tissue for detection by the ground pad and the current and voltage between the electrodes is determined and use to calculate the electrical impedance radially across the shaded section of tissue.

A method of the invention is described with reference to FIG. 7. In a first step 100, a device of the invention comprising a delivery catheter attached to an implantable occlusion body is advanced into the heart along femoral vein, iliac vein, IVC, right atrium, transeptally to left atrium or alternatively the brachial vein, jugular vein, right atrium, transeptally to left atrium and the occlusion apparatus is positioned in the mouth of the left atrial appendage (LAA). In a second step 110, the occlusion apparatus is radially deployed to close the mouth of the LAA and bring the circumferential array of electrodes into contact with the wall of the LAA. In a third step 120, the processor then actuates the controller to operate in a sensing modality, measuring the electrical impedance of the tissue along a plurality of sections circumferentially around the wall of the LAA, each section being defined by an electrode pair as described above. In a fourth step 130, the processor compiles an electrical impedance profile comprising the electrical impedance measurements around the wall of the LAA. In a fifth step 140, the processor calculates the pulsed field ablation energy to be applied to electrode pairs based on the electrical impedance profile. In a sixth step, the processor controls the output characteristics of the controller to energise each electrode with PFA in a pattern synergistic with the electrical impedance profile.

### Example 1—Exemplary PFA Profile

Signal generator delivers 10 trains of PFA energy to an electrode.

Each train has 30 pulses

Inter-phase delay of 100 μs.

Inter-pulse delay of 100 μs.

Pulse width of 100 μs.

Voltage amplitude of 1000 V.

### Example 2—Treatment of Left Atrial Appendage with Pulsed Field Ablation Employing Circumferential Electrical Impedance Measurements

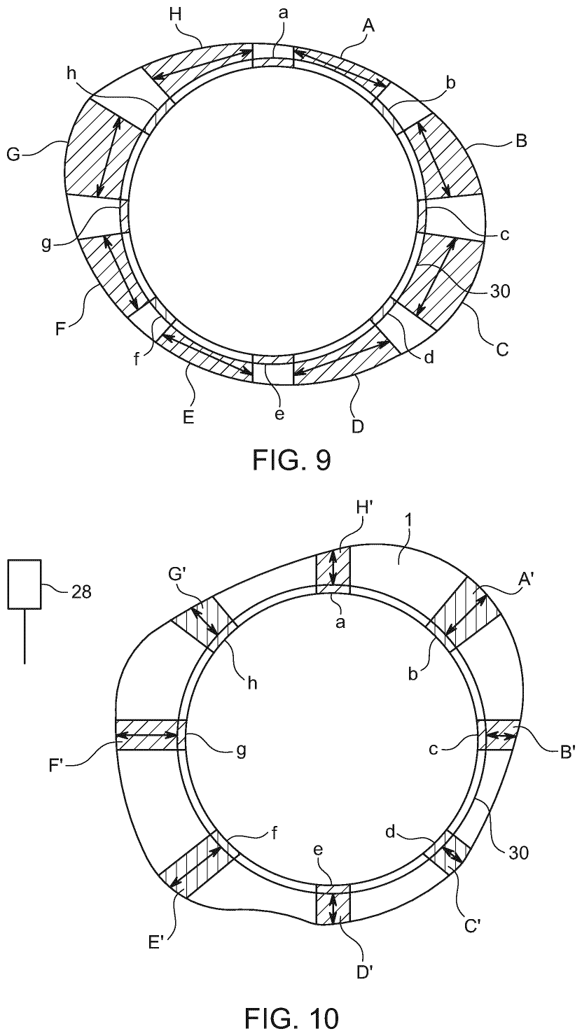

Referring to FIG. 9, an occlusion apparatus 30 has an array of eight electrodes a to h circumferentially arranged the wall of the occlusion apparatus. The electrical impedance profile circumferentially across sections of the wall 1 of the left atrial appendage (Sections A to H) is determined by actuating the controller to create electrical circuits between the controller and pairs of electrodes to pass an alternating electrical current at a defined frequency across the respective sections and measure the voltage and/or current drop across the section and calculate electrical impedance of the tissue. The electrical impedance of the tissue sections were determined to be:
Section A (between electrode a and b)—low electrical impedance
Section B (between electrode b and c)—low electrical impedance
Section C (between electrode c and d)—high electrical impedance
Section D (between electrode d and e)—low electrical impedance
Section E (between electrode e and f)—low electrical impedance
Section F (between electrode f and g)—low electrical impedance
Section G (between electrode g and h)—high electrical impedance
Section H (between electrode h and i)—low electrical impedance

The electrical impedance values were compiled by the processor into an electrical impedance profile which was used to calculate output characteristics for the controller so as to energise the electrode pairs differentially around the circumference of the LAA, where lower PFA power was applied to sections C and G and higher PFA power was applied to sections A, B, D, E, F and H. This results in a non-uniform e-field generated around the circumference of the LAA helping avoid over-treating areas of the tissue that have low electrical impedance.

### Example 3—Treatment of Left Atrial Appendage with Pulsed Field Ablation Employing Radial Electrical Impedance Measurements

Referring to FIG. 10, an occlusion apparatus 30 has an array of eight electrodes a to h circumferentially arranged around the wall of the occlusion apparatus. A ground pad 28 is also shown which in use is attached to the leg of the patient. The electrical impedance profile across radial sections of the wall 1 of the left atrial appendage (Sections A to H) is determined by by actuating the controller to create electrical circuits between the controller and a single of electrode on the occlusion apparatus and the ground pad 28 placed typically on the leg of the subject to pass an alternating electrical current across the respective sections and measure the voltage and/or current drop across the section and calculate electrical impedance of the tissue. The electrical impedance of the tissue sections were determined to be:
Section A′—high electrical impedance
Section B′—low electrical impedance
Section C′—low electrical impedance
Section D′—low electrical impedance
Section E′—high electrical impedance
Section F′—high electrical impedance
Section G′—low electrical impedance
Section H′—low electrical impedance

The electrical impedance values were compiled by the processor into an electrical impedance profile which was used to calculate output characteristics for the controller so as to energise the electrode pairs differentially around the circumference of the LAA, where lower PFA power was applied to sections defined between electrodes a-b, b-c, f-g and g-h and higher PFA power was applied to sections defined between electrodes c-d, d-e and e-f. This results in a non-uniform e-field generated around the circumference of the LAA helping avoid overheating areas of the tissue that have low electrical impedance.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A device (10) to occlude the left atrial appendage (1) of a heart of a subject, comprising:
an implantable occlusion apparatus (30) configured for radial expansion upon deployment to fluidically occlude the left atrial appendage;
an elongated catheter member (80) having a distal end attachable to the implantable occlusion apparatus for transluminal delivery of the implantable occlusion apparatus to the left atrial appendage;
a tissue energising module (20) having a plurality of electrodes (26) disposed around a circumference of the implantable occlusion apparatus in which each electrode is configured to contact a wall of the left atrial appendage at a tissue focal point upon deployment of the implantable occlusion apparatus; and
an electrical controller (40) including a pulsed field energy delivery generator operably attachable to an electrical power source (50) and the plurality of electrodes (26) and configured to energise the electrodes in a pulsed field ablation modality, in which the electrical controller (40) is configured to energise the plurality of electrodes (26) independently to apply a non-uniform pulsed field ablation treatment circumferentially around the wall of the left atrial appendage
characterised in that the device comprises a processor (60) operably coupled to the electrical controller (40) and configured to generate an electrical parameter profile of the LAA comprising electrical parameter measurements taken at a plurality of sections around the circumference of the wall of the LAA and modify the output of the electrical controller so as to independently energise the electrodes (26) or sets of electrodes in a pattern synergistic with the tissue parameter profile to energise some electrodes or sets of electrodes with greater ablative power than others to create a non-uniform tissue ablation circumferentially around the wall of the LAA, tuned to the specific anatomy of the subject.

2. A device according to claim 1, comprising a tissue parameter sensor configured to obtain the electrical parameter measurements.

3. A device according to claim 2 in which the electrical parameter of the tissue is electrical impedance.

4. A device according to claim 3, in which the tissue parameter sensor is configured to measure an electrical parameter of the tissue at a plurality of circumferential or radial sections around the wall of the LAA.

5. A device according to claim 4, in which the tissue parameter sensor comprises the electrical controller (40) and the tissue energising module (20), and in which the processor is configured to actuate the controller and tissue energising module in at least two separate modalities selected from:
a tissue ablation modality configured to deliver a pulsed field ablation treatment at one or more sections around the circumference of the wall of the LAA; and
a tissue parameter measurement modality to measure an electrical parameter of tissue at the plurality of sections around the circumference of the wall of the LAA.

6. A device according to claim 5, in which the processor is configured to (a) compare the electrical parameter measurement for a section of the wall with a reference electrical parameter value, (b) calculate a pulsed field ablation power to be delivered to the section based on the comparison, and (c) modify the output characteristics of the electrical controller to independently energise selected electrodes to deliver pulse field ablation therapy to the section of tissue at the calculated power.

7. A device according to claim 6, in which the processor is configured to perform steps (a), (b) and (c) for each of a plurality of sections of the wall of the LAA.

8. A device according to claim 7, in which the plurality of sections together make up a full circumference of the LAA.

9. A device according to claim 8, in which the controller is configured to measure electrical impedance across a section of the wall of the LAA by configuring one pair of electrodes to supply measuring current and a separate pair of electrodes to detect voltage drop produced by current flowing across the section of tissue.

10. A device according to claim 9, having at least four electrodes circumferentially spaced around the wall of the occlusion apparatus.

11. A device according to claim 10, having six to twelve electrodes circumferentially spaced around the wall of the occlusion apparatus.

12. A device according to claim 11, in which the device is configured to:
deliver by the controller and electrodes a pulsed field ablation treatment at a section of the wall of the body lumen,
determine by the controller and electrodes an electrical impedance value of the tissue at the treated section; and
correlate by the processor an output indication of electrical isolation of the section of the wall of the body lumen based on the electrical impedance value.

13. A device according to claim 12, in which the processor is configured to receive as an input the measured electrical impedance value for the section of tissue, compare the value with one or more reference electrical impedance values, and calculate an output indication of electrical isolation of the section of the wall based on the comparison.

14. A device according to claim 11, in which the device is configured to:
determine by the controller and electrodes a first electrical parameter value selected from an electrical impedance or electrical activity of tissue at a section of the wall of the body lumen,
deliver by the controller and electrodes a pulsed field ablation treatment at the section of the wall of the body lumen,
determine by the controller and electrodes a second electrical parameter value selected from an electrical impedance or electrical activity of the tissue at the treated section; and
calculate by the processor an output indication of electrical isolation of the section of the wall of the body lumen based on a comparison of the first and second electrical parameter measurements.

15. A device according to claim 14, including an earth electrode configured for attachment to a surface of the subjects body, in which the processor is configured to determine the electrical parameter of the tissue at a specific location using an electrode of the tissue energising module and the earth electrode.

16. A device according to claim 15, in which the pulsed field energy delivery generator is configured to deliver at least one pulse train of energy to the tissue of the body lumen, each pulse train of energy including:
at least 60 pulses,
an inter-phase delay between 0 μs and 5 μs,
an inter-pulse delay of 2 to 500 μs,
a pulse width of 1 to 15 μs, and
a voltage of 500 to 2500 V.

17. A device according to claim 16, in which the generator is configured to deliver at least one pulse train of energy with inter-pulse delay is about 2 to about 400 μs.

18. A device according to claim 16, in which the generator is configured to deliver at least one pulse train of energy with inter-pulse delay is about 2 to about 250 μs.
